(19) United States
(12) Patent Application Publication
(10) Pub. No.: US 2026/0273508 A1
(43) Pub. Date: Sep. 17, 2026

(54) CHROMATOGRAPHY MEDIUM AND PROCESS

(71) Applicant: Cytiva BioProcess R&D AB, Uppsala (SE)

(72) Inventors: Matthew Miell, London Greater London (GB); William Law, Stevenage Hertfordshire (GB); Helen Cheek, Stevenage Hertfordshire (GB); Susana Brito Dos Santos, Stevenage Hertfordshire (GB); Daniel McNerney, Stevenage Hertfordshire (GB); Peter Guterstam, Uppsala (SE); Jonathan Behrendt, Stevenage Hertfordshire (GB); Frederic Leroux, Stevenage Hertfordshire (GB); Valeria Mastrullo, Amersham Buckinghamshire (GB)

(21) Appl. No.: 19/166,827

(22) PCT Filed: Mar. 15, 2024
(86) PCT No.: PCT/EP2024/056909

§ 371 Date: Sep. 18, 2025

(30) Foreign Application Priority Data
Mar. 20, 2023 (GB) 2303986.0
Jan. 9, 2024 (WO) PCT/EP2024/050360

(51) Int. Cl. B01J 20/289; B01J 20/24; B01J 20/26; B01J 20/28; C12N 15/10

(52) U.S. Cl. CPC B01J 20/289; B01J 20/24; B01J 20/265; B01J 20/28023; C12N 15/101; B01J 2220/54

(57) ABSTRACT

A chromatography medium for separating an analyte in a solution is provided, the chromatography medium comprising a matrix material having a mean flow pore size of 0.1-2.0 μm, wherein the matrix material is functionalized with a thiophilic aromatic ligand to a ligand concentration up to 1500 μmol/g matrix material. Provided are also processes of separating an analyte in a solution using the chromatography medium. The invention is useful for separating different isoforms of plasmid DNA.

### TECHNICAL FIELD

The present disclosure relates to a chromatography medium for separating an analyte in a solution and to a process of separating an analyte using such chromatography medium.

### BACKGROUND ART

Plasmids are raw material in mRNA and viral vector manufacturing, hence why there is a demand for highly purified plasmid DNA. In purification of supercoiled plasmid DNA, a complete removal of other cell components such as host proteins, endotoxins, chromosomal DNA, RNA, open circular and nicked forms of plasmid DNA is necessary.

Different chromatographic methods have been used for plasmid DNA purification, such as size exclusion chromatography, gel filtration, hydroxyapatite, ion exchange chromatography, reversed phase chromatography and hydrophobic interaction chromatography. Most of the methods lack the possibility to separate supercoiled plasmid DNA from other forms of the plasmid.

R. Lemmens et al., J. Chromatogr. B 784 (2003) 291-300 describes a three-step purification process for purifying plasmid DNA, comprising i) group separation, ii) thiophilic aromatic chromatography using various thioether containing adsorbents coupled to Sepharose 6 Fast Flow chromatography resin, and iii) anion-exchange chromatography.

There is, hence, a need for an improved method for separation of isoforms of plasmid DNA.

### SUMMARY OF THE INVENTION

It is an object of the present invention to provide a chromatography medium for separating an analyte in a solution, such as supercoiled plasmid DNA. It is also an object to provide a process for separating an analyte in a solution.

The invention is defined by the appended independent patent claims. Non-limiting embodiments emerge from the dependent patent claims, the appended drawings and the following description.

According to a first aspect there is provided a chromatography medium for separating an analyte in a solution, the chromatography medium comprising a matrix material having a mean flow pore size of 0.1-2.0 μm, wherein the matrix material is functionalized with a thiophilic aromatic ligand to a ligand concentration up to 1500 μmol/g matrix material.

As used herein, the term “thiophilic” refers to the interaction between the ligand and the target which is based on thiophilic aromatic chromatography. The ligand concentration may be 10-1500 μmol/g, or 50-1400, or 100-1300, or 150-1200, or 200-1100, or 250-1000, or 300-1000, 400-1000, or 500-1000, or 600-1000, or 700-1000, or 800-1000, or 300-900 μmol/g of matrix material.

The analyte may be plasmid DNA or any polynucleotide such as RNA, genomic DNA, sheared nucleotides sequences, etc. The analyte may preferably be less than 20 kbp in size. In particular, the analyte may be a specific isoform of plasmid DNA.

Standard resins used in chromatography have a limited pore access for large entities, such as plasmids, and are diffusion-dependent. The present chromatography medium comprising a matrix material, with a mean flow pore size of 0.1-2.0 μm and functionalized with a thiophilic aromatic ligand, allows for higher flow rates, which translates into better processing times. There is further an increased binding capacity (e.g. >5 times higher) using the present medium compared to standard resins. This matrix material gives a high capacity for separation of large analytes/entities, such as plasmids.

The matrix material may be a convective flow material, where a flow through such materials is convective rather than diffusional. A convective flow matrix material includes any matrix in which application of a hydraulic pressure difference between the inflow and outflow of the matrix forces perfusion of the matrix, achieving substantially convective transport of the substance(s) into the matrix or out of the matrix, which is effected very rapidly at a high flow rate.

Examples of convective matrix materials include porous adsorptive membranes and monolithic materials. The adsorptive membrane can for example be a polymeric membrane, such as a membrane comprising polyether sulfone. Another example of an adsorptive membrane is a polymer nanofiber membrane, such as for example cellulose, cellulose acetate and cellulose fibers, optionally treated or derivatized for use as an adsorbent. A polymer used for a polymeric membrane or polymer nanofiber membrane may be a natural or synthetic polymer, including a derivatized polymer, such as a hydroxylated polymer. The adsorptive membrane could alternatively be a monolithic material or a conventional membrane made by emulsification. Another alternative is a 3D printed material.

With the present matrix material there is no reliance on diffusion of the analyte into pores. With the present matrix material a residence time counted in seconds is sufficient, and hence quick loading of low-titre samples is made possible. The analyte is attracted to the thiophilic aromatic ligand of the functionalized matrix material and bound thereto. The use of the thiophilic aromatic ligand enables purification of for example plasmids, such as supercoiled plasmid DNA, for use in gene therapy, DNA vaccines and research applications.

The matrix material may be a fibrous material. The fibrous material may be a non-woven polymer material. The matrix material could comprise or consist of cellulose and/or other polymers beyond cellulose. A fibrous cellulose matrix material may be produced through electrospinning of cellulose acetate that has undergone multiple reaction steps.

The thiophilic aromatic ligand may be an amine-derived aromatic ligand. The amine derived aromatic ligand may comprise an aromatic ring having at least one nitrogen atom as member of the ring.

The ligand may be comprise a thioether moiety connected to an aromatic ring, the aromatic ring preferably containing at least one nitrogen atom as a member of the ring.

The thiophilic aromatic ligand may comprise an aryl group selected from pyridyl, phenyl, benzyl, toluyl, phenethyl, naphtyl, imidazolyl, pyrazolyl, pyrazinyl, pyrimidinyl, pyridazinyl, piperidinyl, morpholinyl, piperazinyl, indolyl, quinolinyl, purinyl groups and any substituted such group.

Such substituted group may for example be a substituted phenyl group having one or more electron withdrawing groups. One example is nitrophenyl. The electron withdrawing group may be —CF₃, or may be a group comprising nitrogen, such as —NO₂ or —CN.

The thiophilic aromatic ligand or a portion of the ligand may be described by the formula:

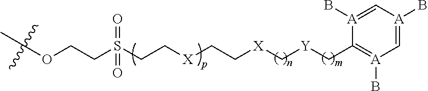

wherein, independently
X for each occurrence is NH, O or S,
Y is S or CH₂,
A is Cor N,
B is H or an electron withdrawing group, or is absent,
n is 0 or an integer 1-12,
m is 0 or an integer 1-12, and
p is 0 or an integer 1-12.

The electron withdrawing group may be —CF₃, or may be a group comprising nitrogen, such as —NO₂ or —CN.

In embodiments where A is N, B may be absent.

Alternatively, the matrix material M may be functionalized with a ligand according to the formula:

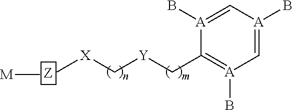

wherein, independently
M represents a matrix material,
Z represents a linker, optionally including a spacer moiety,
X for each occurrence is NH, O or S,
Y is S or CH₂, or is absent,
A is C or N,
B is H, a C1-C6 alkyl group such as a C1-C3 alkyl group, or an electron withdrawing group, or is absent,
n is 0 or an integer 1-12, and
m is 0 or an integer 1-12.

The electron withdrawing group may be —CF₃, or may be a group comprising nitrogen, such as —NO₂ or —CN.

Preferably, at least one occurrence of A is N.

In embodiments where A is N, B may be absent.

The thiophilic aromatic ligand may be selected from 2-mercaptopyridine, 4-mercaptopyridine and 2-mercapto-ethyl-pyridine.

The linker may comprise a moiety resulting from the reaction of coupling the ligand to the matrix material. Well-known coupling chemistries are known to persons of skill in the art and may involve reaction with e.g. divinyl sulfone (DVS) as described herein, or using epichlorohydrin (ECH) or allyl glycidyl ether (AGE). Thus, the linker may contain and be attached to the matrix material through a moiety obtained by reaction of the matrix material, optionally activated or derivatised, with divinyl sulfone, by reaction with epichlorohydrin, or by reaction with allyl glycidyl ether. For example, the linker may comprise a vinyl sulfone moiety.

Optionally, the linker Z may also comprise a spacer or extender moiety as described elsewhere herein.

Optionally, the ligand may be connected via an oxygen to the matrix material.

The ligand may be connected to the matrix material through an extender group selected from polysaccharide structures and polymeric structures. The extender builds off of the substrate through polymerisation (undetermined length).

According to a second aspect there is provided a chromatography unit comprising the chromatography medium described above.

The chromatography unit is suitable for use in chromatography. Typically, the chromatography unit comprises one or more solution inlets and outlets. In the chromatography unit, the chromatography medium is arranged such that the solution is passed though the medium. By means of a pump connected to the chromatography unit, a solution flow rate may be created through the medium in the unit.

The chromatography unit may be a sanitized/cleaned unit, preferably compatible with Good Manufacturing Practice (GMP) which entails having been exposed to up to 4 hrs in 0.5 M NaOH and 40% isopropyl alcohol, thus reducing the bioburden and endotoxin level of the unit.

In a further aspect of the invention, the chromatography medium or the chromatography unit may be used for separation of different isoforms of plasmid DNA present in a solution, such as for the purification of supercoiled plasmid DNA.

According to a further aspect there is provided a process of separating an analyte in a solution, the process comprising: obtaining a solution comprising an analyte, adding the solution to the chromatography medium described herein in a binding buffer having a conductivity of 240-270 mS/cm, eluting the analyte from the chromatography medium by contacting the chromatography medium with an elution buffer having a conductivity of 0-240 mS/cm, and collecting the thus formed eluate containing the analyte.

The solution may for example comprise different plasmid DNA (pDNA) isoforms, e.g. supercoiled plasmid DNA, open circular and nicked forms of plasmid DNA, wherein the analyte of interest typically is the supercoiled plasmid DNA isoform.

The binding buffer allows or facilitates binding of the analyte to the ligand on the matrix material of the medium. The conductivity chosen, 240-270 mS/cm, depends on the characteristics of the target analyte. Eluting the analyte from the medium is performed by using an elution buffer having a lower conductivity than the binding buffer, 0-240 mS/cm. The elution may be a stepwise of gradient elution in which the chromatography medium is contacted with elution buffers of decreasing conductivity.

The conductivity of the binding buffer may be optimized such that the unwanted plasmid DNA isoforms do not bind to the unit and are collected in a flow-through fraction together with RNA and other impurities, while the analyte of interest, the supercoiled plasmid DNA, is retained in the chromatography medium by binding to the ligand and is subsequently eluted with the elution buffer. Such a single elution step process may be favourable in terms of yield.

According to yet another aspect there is provided a process of separating different isoforms of plasmid DNA in a solution, the process comprising obtaining a solution comprising isoforms of plasmid DNA in a solution, adding the solution to the chromatography medium described above in a binding buffer having a conductivity of 240-270 mS/cm, contacting the chromatography medium with a first elution buffer having a conductivity of 200-240 mS/cm, collecting the thus formed eluate containing a first plasmid DNA isoform, and thereafter contacting the chromatography medium with a second elution buffer having a conductivity of 0-200 mS/cm, and which conductivity is lower than the conductivity of the first elution buffer, collecting the thus formed eluate containing a second plasmid DNA isoform.

The solution may comprise different plasmid DNA isoforms, e.g. supercoiled plasmid DNA, open circular and nicked forms of plasmid DNA, and impurities such as RNA. The analyte of interest is typically the supercoiled plasmid DNA isoform. The first eluate contains the first plasmid DNA isoform, which comprises the wanted purified supercoiled plasmid DNA. The second eluate contains the unwanted plasmid DNA isoforms, such as open circular and/or nicked forms of plasmid DNA, together with RNA and other impurities. This two-step process may hence be used to separate the plasmid DNA isoforms, wherein the first step is the wanted product and the second is everything else. Such a process may be used on an industrial scale.

The binding buffer used should promote binding of the analyte to the ligand on the matrix material of the medium. The conductivity of the binding buffer may be greater than 200 mS/cm, such as 240-270 mS/cm, and the conductivity chosen depends on the characteristics of the target analyte. Examples of binding buffers are: sodium sulfate, ammonium sulfate (base buffers), TE buffer: 10-20 mM Tris/1 mM EDTA/2.0-2.4 M ammonium sulphate, a buffer comprising: 2.4-2.0 M ammonium sulphate/0.3 M NaCl/100 mM Tris/10 mM EDTA.

Eluting the analyte from the chromatography medium may comprise a stepwise or gradient elution in which the chromatography medium is contacted with elution buffers of decreasing conductivity.

The elution buffer used should elute the analyte from the medium. The conductivity of the elution buffer may be 0-240 mS/cm, and the choice of elution buffer depends on analyte recovery and analyte stability in solution. Some analytes may require a specific salt to remain stable. Examples of elution buffers include a buffer comprising: 10-20 mM Tris/1 mM EDTA/1.7 M ammonium sulphate. Buffers having lower or no ammonium sulphate content (including water alone) can also strip bound components.

When a first and second elution buffer are used to elute plasmid DNA from the chromatography medium, the chromatography medium is first contacted with a first elution buffer having a conductivity of 200-240 mS/cm. Such a first elution buffer may for example be: 1.7 M ammonium sulphate/0.3 M NaCl/100 mM Tris/10 mM EDTA. After collection of the formed eluate containing a first plasmid DNA isoform, the chromatography medium is contacted with a second elution buffer having a conductivity of 0-200 mS/cm, and which conductivity is lower than the conductivity of the first elution buffer, and the thus formed eluate containing a second plasmid DNA isoform is collected.

By such a process e.g. supercoiled DNA may be purified and enriched by greater than 90% from the starting solution.

The process may further comprise, after the eluting step, a step of adding the eluate to a multimodal chromatography resin.

The process may further comprise cleaning of the chromatography medium after collecting the eluate, the cleaning comprising to add a solution of 0.1-2M NaOH to the chromatography medium. Alternatively, cleaning of the chromatography medium after collecting the eluate, may comprise to add a solution of 0.1-0.5 M HCl or phosphoric acid to the chromatography medium. Cleaning ensures that the chromatographic medium can be used for multiple chromatographic runs.

### BRIEF DESCRIPTION OF THE DRAWINGS

FIGS. 1a-1d illustrate different reaction schemes for preparing a matrix material for a chromatography medium. The matrix material comprising a fibrous non-woven polymer material comprising fibers with vinylsulfone reactive groups functionalized with a thiophilic aromatic ligand. FIG. 1a: addition of 2-((2-(pyridin-2-yl)ethyl)thio)ethan-1-amine dihydrochloride to an activated crosslinked substrate; FIG. 1b: addition of cystamine dihydrochloride to the substrate, followed by a reduction with dithiothreitol, and thereafter addition of 2-vinylpyridine; FIG. 1c: addition of 2,2′-(ethylenedioxy)diethanethiol to an activated cross-linked substrate followed by 2-vinylpyridine; FIG. 1d: addition of di(2-mercaptoethyl) ether to an activated cross-linked substrate followed by addition of 2-vinylpyridine.

FIG. 2 is a bar chart showing the dynamic binding capacity for each of the routes illustrated in FIGS. 1a-1d. Routes 1a to 1d result in similar dynamic binding capacity (determined by QB10 method (dynamic binding capacity at 10% breakthrough volume (QB10)), however, routes 1c and 1d showed a higher recovery (100%) compared to route 1a and 1b.

FIG. 3 shows variants of thiophilic aromatic ligands used for functionalising the matrix material.

FIG. 4 shows a chromatogram demonstrating the separation of plasmid DNA from RNA in a solution, using a matrix material functionalized with a thiophilic aromatic ligand at a ligand concentration of 900 μmol/g matrix material, and using a flow rate of 5 MV/min (Membrane Volumes per minute).

FIG. 5 illustrates size ranges for plasmid purification and recovery using matrix materials with three different ligand densities and a range of differently sized plasmids.

FIG. 6 shows an agarose gel loaded with sample from the wash (A3 W in FIG. 6), the elution (A3 E in FIG. 6) and the strip phases (A3 S in FIG. 6) when purifying plasmid DNA using a matrix material functionalized with a thiophilic aromatic ligand at a ligand concentration of 900 μmol/g matrix material. The gel was also loaded with a sample filtered with tangential flow filtration (TFF, with a 300 kDa cut-off) (Post TFF1 in FIG. 6), and with a sample as added to the chromatography column (PS Load in FIG. 6).

FIGS. 7a-7b show bar charts showing the quantity (%) of each impurity in each phase: wash, elution, and strip.

### DETAILED DESCRIPTION

Plasmids are raw material in mRNA and viral vector manufacturing, why there is a demand for highly purified plasmid DNA. Supercoiled plasmid DNA may be expressed in cells of any origin. Most commonly, microorganisms like bacteria, such as E. coli, are used for culturing the plasmids, but the use of host cells is not limited and can be prokaryotic or eukaryotic cells. The host cells harbouring the plasmid can be cultivated in a number of ways well known in the art, e.g. in incubator, bioreactor, fermenter etc. The plasmid can be of virtually any size, e.g. in the range of 1 kb up to 500 kb. The plasmids can be of a high copy number or low copy number and can carry any gene, either genomic or synthetic, encoding protein or peptide of interest, from any source. The culturing of the host cells, as well as the exploitation of the plasmid for gene therapy, is well known in the state of the art.

After culturing the host cells containing the plasmid, the cells are recovered by e.g. centrifugation or filtration. The cells can be stored, for example in a freezer, or processed immediately. After a step of disintegrating the cells, performed e.g. by lysis, such as alkaline lysis, a solution comprising the supercoiled plasmids and any impurity, e.g. open circular and nicked plasmids, RNA, host proteins, endotoxins, and chromosomal DNA, is obtained.

In purification of supercoiled plasmid DNA, a complete removal of other cell components such as host proteins, endotoxins, chromosomal DNA, RNA, open circular and nicked forms of plasmid DNA is necessary. Separation of different plasmid DNA isoforms is a major challenge in purifying plasmid DNA.

The term “supercoiled plasmid DNA” refers to plasmid DNA in the form of a closed loop, which has a coiled topology. DNA may be positively or negatively supercoiled. For example, supercoiled plasmid DNA may form a two-start right-handed helix with terminal loops.

The term “open circular plasmid DNA” refers to plasmid DNA in which one of the strands of the DNA double helix has been broken, with the effect that at least part of the strain on the double helix, that causes plasmids to assume a supercoiled configuration, has been released. Thereby, open circular pDNA forms a closed loop which is not coiled, but maintains a circular topology.

Below is described a chromatography medium for separating analytes in a solution, such as plasmid DNA or any polynucleotide such as RNA, genomic DNA, sheared nucleotide sequences, etc. The use of the present chromatography medium may facilitate the production of highly purified supercoiled plasmid DNA for use in gene therapy and DNA vaccine applications.

The matrix material of the chromatography medium used is a material having a mean flow pore size of 0.1-2.0 μm, wherein the matrix material is functionalized with a thiophilic aromatic ligand to a ligand concentration up to 1500 μmol/g of matrix material.

The matrix material may be a convective flow matrix material. Such a material may for example be an adsorptive membrane where a flow through such materials is convective rather than diffusional. A convective matrix material includes any matrix in which application of a hydraulic pressure difference between the inflow and outflow of the matrix forces perfusion of the matrix, achieving substantially convective transport of the substance(s) into the matrix or out of the matrix, which can be effected very rapidly at a high flow rate.

Examples of convective matrix materials include porous adsorptive membranes and monolithic materials. The adsorptive membrane can for example be a polymeric membrane, such as a polyether sulfone membrane, a polymer nanofiber membrane, such as for example cellulose, cellulose acetate and cellulose fibers, which have been treated for use as an adsorbent. Treatment may include one or more of cross-linking, derivatization, and coupling of a ligand. The matrix material may be a non-woven material comprising fibers, such as from cellulose. Such fibrous substrate may be based on electrospun polymeric fibers or cellulose fibers. and may e.g. have a cross-sectional diameter of 10-1000 nm, such as 200-800 nm, 200-400 nm or 300-400 nm. Such a matrix material can be found in a HiTrap Fibro unit from Cytiva, Sweden.

A polymer used for a polymeric membrane or polymer nanofiber membrane may be a natural or synthetic polymer, including a derivatized polymer. For example, a hydroxylated polymer may be used, such as to provide a hydroxylated polymeric membrane. A hydroxylated polymer may include a glycopolymer. In addition to cellulose and polyether sulfone as mentioned above, polymers useful in adsorptive membranes include polymers based on polytetrafluoroethylene (PTFE), polypropylene, polyamide or polyacrylamide.

The adsorptive membrane could alternatively be a monolithic material or a conventional membrane made by emulsification. Another alternative is a 3D printed material.

Mean flow pore (MFP) size is an indicator of material flow characteristics, and is measured by capillary flow porometry, based on the displacement of a wetting liquid with a known surface tension from the sample pores by applying a gas at increasing pressure. The higher the MFP size, the larger the flow of liquid through the material at a given pressure. The mean flow pore size is calculated from the point at which 50% of the flow goes through a sample. Mean flow pore size thus corresponds to the pore size calculated at the pressure where the wet curve and the half-dry curve meet.

In an alternative definition, the mean flow pore size of the present matrix material may be seen as an effective pore size defined as the size of the largest sphere that is able to pass through the pore.

The mean flow pore size of the matrix material may be 0.1-2.0 μm, 0.1-1.8 μm, 0.1-1.6 μm, 0.1-1.4 μm, 0.1-1.2 μm, 0.1-1.0 μm, 0.1-0.8 μm, 0.1-0.6 μm, 0.1-0.4 μm, 0.1-0.2 μm, 0.2-2.0 μm, 0.4-2.0 μm, 0.6-2.0 μm, 0.8-2.0 μm, 1.0-2.0 μm, 1.2-2.0 μm, 1.4-2.0 μm, 1.6-2.0 μm, 1.8-2.0 μm, or 0.5-1.5 μm.

The matrix material may be functionalized with ligands to a ligand concentration of 10-1500 μmol/g matrix material. The matrix material may be functionalized with a thiophilic aromatic ligand to a ligand concentration of 10-1500 μmol/g matrix material. Under well-defined conditions, these ligands are capable of separating supercoiled plasmid DNA from its open circular isoform.

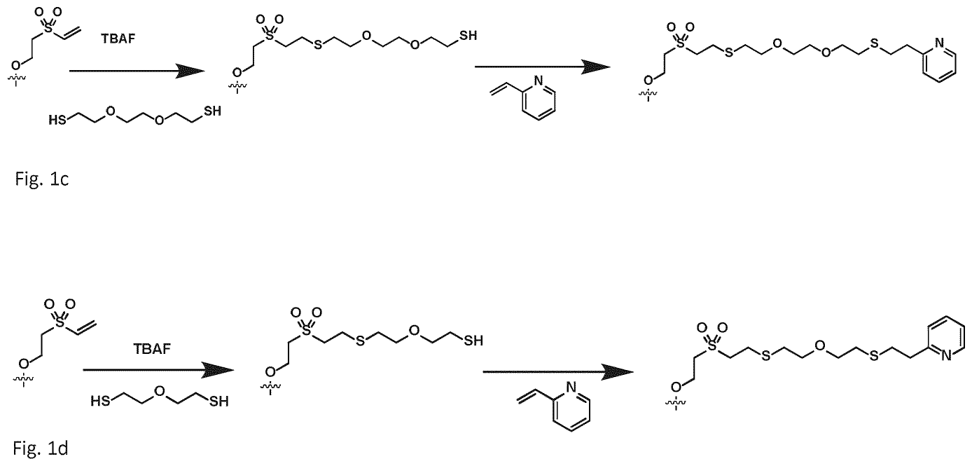

The thiophilic aromatic ligand, see FIG. 3, may comprise an aryl group selected from pyridyl, phenyl, benzyl, toluyl, phenethyl, naphtyl, imidazolyl, pyrazolyl, pyrazinyl, pyrimidinyl, pyridazinyl, piperidinyl, morpholinyl, piperazinyl, indolyl, quinolinyl, purinyl groups, and substituted such groups. Such substituted groups may for example be substituted phenyl groups having one or more electron withdrawing groups. One example is nitrophenyl. In examples, the thiophilic aromatic ligand may be 2-mercapto-ethyl-pyridine.

The thiophilic aromatic ligand may be connected to the matrix material through an extender group selected from polysaccharide structures and polymeric structures. The extender group may be e.g. dextran, acrylamides or polyglycerol. If dextran is used as an extender it may have a molecular weight in a range of from 5,000 to 2,000,000 Dalton. The extender group builds off of the substrate through polymerisation (undetermined length).

In some embodiments, no extender group is used, and the ligand is immobilized/connected to the matrix material directly using for example a linker, such as a crosslinker. The linker can be formed by the reagent used to activate the matrix material and that allows the coupling of the ligand. Well-established methods include for example the introduction of epoxy groups via the use of epichlorohydrin or 1,4-butanediol diglycidyl ether. Another example is the introduction of double bounds on the matrix material with the use of reagents such divinyl sulfone. The crosslinker composed by these reagents can be further extended by a new functionalisation that would allow the coupling of the ligand.

Spacers, such as 2-12 carbon alkyls, linear or branched, or 2-12 carbon ethers, can be used between the thiophilic aromatic ligand and a crosslinker, such as vinyl sulfone. The linker, crosslinker, may also react with an extender group and link it to the ligand or spacer. The spacer is the length of carbons between the linker and the ligand used to improve the base stability of the linker-ligand bond.

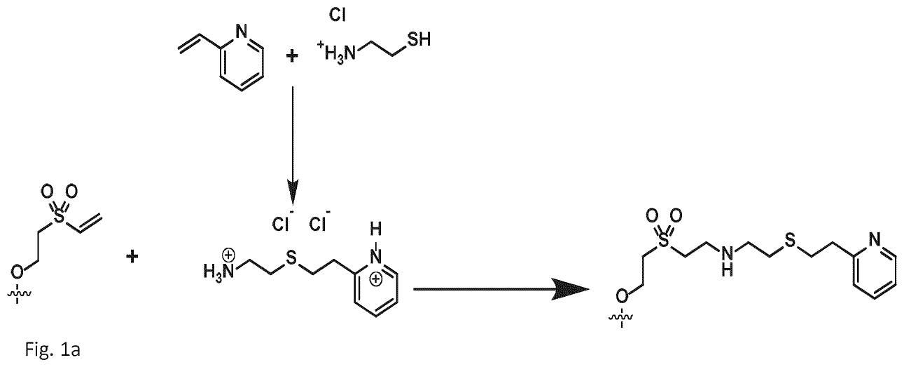

FIGS. 1a-1d illustrate different exemplifying reaction schemes for functionalizing a fibrous non-woven polymer material comprising fibers with vinylsulfone reactive groups. The thus formed matrix material comprises vinylsulfone reactive groups functionalized with a thiophilic aromatic ligand.

FIG. 1a illustrates that 2-((2-(pyridin-2-yl)ethyl)thio)ethan-1-amine dihydrochloride (0.14 M) in water is allowed to react at pH 9.6 with a fibrous non-woven polymer material comprising fibers with vinylsulfone reactive groups to form functionalized matrix material with thiophilic aromatic ligand.

FIG. 1b illustrates an exemplifying reaction scheme of functionalizing a fibrous non-woven polymer material comprising fibers crosslinked and activated with divinyl sulfone with an aqueous solution of cystamine dihydrochloride (0.22 M) at pH 9, followed by reduction of the disulfide bond with an aqueous solution of dithiothreitol (0.66 M) to give a cysteamine-derivatised substrate. The thus obtained exposed sulfhydryl on the substrate is further reacted with 2-vinyl pyridine (30 mM) to form functionalized matrix material with thiophilic aromatic ligand. In FIG. 1c is illustrated an exemplifying reaction scheme of functionalizing a fibrous non-woven polymer material comprising fibers crosslinked and activated with divinyl sulfone with an aqueous solution of 2,2′-(ethylenedioxy)diethanethiol (0.15 M) followed by reaction of the terminal sulfhydryl with 2-Vinylpyridine (30 mM) to form a functionalized matrix material with thiophilic aromatic ligand.

FIG. 1d illustrates an exemplifying reaction scheme of functionalizing a fibrous non-woven polymer material comprising fibers crosslinked and activated with divinyl sulfone with an aqueous solution of di(2-mercaptoethyl)ether (0.15 M) followed by reaction of the terminal sulfhydryl with 2-vinylpyridine (30 mM) to form a functionalized matrix material with thiophilic aromatic ligand.

A solution comprising the analyte, such as plasmid DNA, is added to the functionalized matrix material of the chromatography medium in a binding buffer.

The analytes are attracted to the thiophilic aromatic ligands of the functionalized matrix material and bound thereto.

Washing may be performed after the adsorption but before elution, as is well known in the art, in order to remove retained undesired material.

When the analyte has adsorbed to the ligand, possibly followed by a washing step, elution is performed. Elution may be performed by contacting the chromatography medium with an elution buffer and the thus formed eluate containing analyte is collected. Elution may be performed by using a conductivity gradient with an aqueous solution having a decreasing salt concentration (stepwise or gradient elution).

The elution step can be performed as a dynamic or batch procedure. Elution is conveniently performed according to well-known principles, such as by a gradient of decreasing conductivity.

Typically, elution of the plasmid DNA from the chromatography medium having a matrix material being a convection-based fibrous substrate, such as a fibrous non-woven polymer matrix, can be made at flowrates down to a few seconds residence time, i.e. 60 MV/min, and if required up to several minutes residence time (max 6 min) 0.2 MV/min. Optimal residence time for such fibrous matrix materials is 5-20 MV/min. For resins typical residence times are 1-8 min and the optimum is often at 4 min residence time.

After collecting the eluate, the chromatography medium may be cleaned using 0.5M NaOH, up to 2M NaOH solution, before being used again. Acidic solution is also an option for cleaning in place to reduce any built up foulant. Due to the low residence time (6-12 seconds), only short cleaning holds per cycle are required, as short as one minute.

Standard resins used in chromatography have a limited pore access for large entities, such as plasmids, and are diffusion-dependent. The present chromatography medium comprising a matrix material of non-woven fibrous material functionalized with a thiophilic aromatic ligand allows for higher flow rates, which translates into better processing times. There is further an increased binding capacity (>5 times higher) using the present medium compared to standard resins. This matrix material gives a high capacity for large analytes/entities, such as plasmids. There is no need for diffusion into pores and there is a residence time in seconds and a quick loading of low-titre samples.

Thus, the method and chromatography material above may be used for purification of for example plasmids for use in gene therapy, DNA vaccines and laboratory studies related to gene therapy.

### Experimental Part

Below, the present chromatography material and use thereof will be described by way of examples provided only as an illustration and not to be construed as limiting the scope of the invention as defined by the appended claims in any way.

### Preparation of Plasmids

Plasmids were prepared using conventional plasmid preparation protocols using alkaline lysis followed by purification using a Sepharose 6FF resin. An E. coli pellet, from DH10b cells, was first dissolved in buffer (10 ml/g pellet, 50 mM Tris, 50 mM glucose, 10 mM EDTA, pH 7.5), and then cells were lysed for a maximum time of three (3) minutes by slow addition of 1 volume of lysis buffer (0.2M NaOH, 1% SDS) during gentle stirring. After three minutes the alkaline lysate was neutralized by addition of 1 volume of neutralization buffer (3M KAc, 2.07 M HAc). The neutralized flocculated lysate was then lifted to surface by addition of ammonium bicarbonate (8 g/L) and allowed to incubate overnight for degassing and formation of a stable flocculate cake. Neutralized lysate was then clarified by depth filtration and subsequently concentrated 15 times on hollow fiber by tangential flow filtration (TFF). Finally, plasmid was purified from RNA and protein debris by group separation using Sepharose 6FF resin.

### Preparation of Matrix Material

A solution of cellulose acetate (CA), with a relative molecular mass of 29,000 g/mol, was dissolved in common solvents prior to electrospinning to produce fibres with diameters ranging between 300-600 nm. Optimised conditions for nanofiber production can be found in, for example, O. Hardick, et al, J. Mater. Sci. 46 (2011) 3890. Sheets of approximately 20 g/m2 material were layered and subjected to a combined heating and pressure treatment.

Strips (100×155 mm2) of the formed CA material were placed in between polypropylene gauze and loaded into a flow reactor. The strips were washed three times for 20 minutes with deionised water and then left to stand in the last water wash for 16 hours.

The CA material was saponificated and extended in situ to form cellulose grafted with branched polyglycerol. The flow reactor was drained from the washing water. Potassium hydroxide (156 g) was dissolved in DI H₂O (3.5 L) in a 15 L container. Glycidol (950 mL) was added to the basic solution and stirred for 4 minutes. The reaction mixture was added to the flow reactor containing the CA sheets. The recirculating pump was switched on and the mixture was recirculated for 6 h. After 6 hrs of recirculation, the pump was stopped, and the flow reactor was drained. The material was washed with 4×6 L of distilled water, each wash lasting for 15 min.

Thereafter, divinylsulfone derivatisation of the polyglycerolated cellulose was performed. In an 8 L beaker/container, deionised water (2.963 L) was added followed by Na₂CO₃ (222 g). The content was stirred until complete dissolution of the base. Acetonitrile (HPLC for gradient analysis; >99.9%; 0.886 L) was added and the mixture was stirred for 2 min before being loaded into the flow reactor. The recirculating pump was switched on and the mixture was recirculated for 2 min before adding the divinylsulfone portion (>99%, 0.684 L). The reaction mixture was recirculated for 6 hrs at room temperature. The reaction mixture was drained and the material was washed by recirculation of 1:1 water/acetone (6 L) at 24-26° C., 4 times for 20 minutes each time. The material was then rinsed by recirculation of distilled water (6 L), twice for 15 mins each time. The material was thereafter used.

Functionalization of Matrix Material with Thiophilic Aromatic Ligand, Route 1a (FIG. 1a)

A sheet of Fibro-VS (fibrous non-woven polymer material comprising fibers with vinylsulfone reactive groups) was placed in a polypropylene tray and covered with 25 ml of a solution of 2-((2-(pyridin-2-yl)ethyl)thio)ethan-1-amine dihydrochloride (0.884 g) in deionised water (25 mL) adjusted to pH 9.6. The tray was then placed on an orbital shaker at low speed for 16 hrs at room temperature. After this time the solution was removed by decantation and the sheet was washed with three times by covering the sheet with deionised water (50 mL), placed on the orbital shaker for 5 min and then the wash solution was removed by decantation. After the final wash, the sheet was removed and excess water was blotted off with blue roll disposable towel.

### Blocking of Vinylsulfone Reactive Groups

A phosphate buffered solution of thioglycerol (2.5 v/v % thioglycerol, pH 8.3) was prepared by dissolving sodium phosphate dibasic dodecahydrate (3.58 g) and disodium EDTA dihydrate (37 mg) in water (95 mL) with stirring. Thioglycerol (2.5 mL) was added and the resulting solution was basified to pH 8.3 using 49% NaOH solution and diluted to 100 ml. The sheet of functionalized material was placed in a tray and submerged in 25 ml of buffered thioglycerol solution. The system was shaken gently on an orbital shaker for 16 hrs (150 rpm) before being washed 3 times in water (50 mL) for 15 mins each on the orbital shaker.

Functionalization of Matrix Material with Thiophilic Aromatic Ligand, Route 1b (FIG. 1b)

A sheet of Fibro-VS material was placed in a polypropylene tray and treated with a solution of cystamine dihydrochloride (1.25 g) in deionised water (25 mL) at a pH adjusted to 9. The tray was gently shaken for 18 hrs at room temperature on an orbital shaker set to 250 rpm. The solution was decanted off and the sheet was rinsed four times with deionised water for 20 min.

Reduction of Fibro-Cystamine to Fibro-Cysteamine with DTT

The Fibro-cystamine sheet was placed in a polypropylene tray and treated with a reducing solution made of dithiothreitol (2.56 g), sodium chloride (781 mg), EDTA (33 mg), NaHCO₃ (749 mg) and Na₂CO₃ (94 mg) in deionised water (25 mL). The tray was sealed and shaken to a gentle sway on an orbital shaker for 4 hrs. The solution was removed and the sheet of material was rinsed with deionised water four times for 20 min.

### Addition of 2-Vinylpyridine to Fibro-Cysteamine

The Fibro-Cysteamine sheet was placed in a polypropylene tray and then treated by addition of 2-vinylpyridine (80 μL) in deionised water (25 mL) to give a 30 mM solution. The sheet was gently shaken for 16 hrs at room temperature. The reaction solution was decanted off and the sheet was rinsed with deionised water two times for 20 min, then 3 times with a 1:1 acetone/water mixture for 20 min then three times with deionised water for 20 min.

Functionalization of Matrix Material with Thiophilic Aromatic Ligand Route 1c (FIG. 1c)

2,2′-(ethylenedioxy)diethanethiol (EDDE; 0.625 mL) was added to 25 mL of DI water in a 100 ml Duran bottle under stirring to give a 2.5% v/v solution. Tetrabutylammonium fluoride (92 mg) was added and the pH was adjusted to 9.6. A sheet of Fibro VS was patted dry on absorbing material and placed into sealable polypropylene tray. The solution of dithiol was added to the sheet, and the tray was sealed and placed in a sealed bag on an orbital shaker in a fumehood for 16 hrs. After this time, the solution was decanted off in bleach, replaced with 50 mL deionised water, and the tray was gently shaken on the orbital shaker for 20 minutes. This rinsing process was repeated four times before removing the last water wash.

### Addition of 2-Vinylpyridine to Fibro-EDDE

A 0.1 M phosphate buffer at pH 8 was prepared by dissolving K2HPO4 (16.73 g) and KH2PO4 (0.523 g) in 900 mL of deionised water then adding deionised water QSAD for 1000 mL. A sheet of Fibro-linker-SH, obtained above, was placed in a polypropylene tray and rinsed twice with the freshly prepared phosphate buffer pH 8 before being patted dry. The sheet was then treated in a fumehood by addition of 25 ml of phosphate buffer pH 8, followed by 80 μl of 2-Vinylpyridine to give a 30 mM solution. The polypropylene tray was closed with a lid then placed in a large zip bag, then sealed. The system was shaken overnight at room temperature (20-23° C.). The reaction solution was decanted in bleach and the sheet was rinsed with DI water (6×20 min) before being patted dry and tested.

Functionalization of Matrix Material with Thiophilic Aromatic Ligand Route 1d (FIG. 1d)

Bis(2-mercaptoethyl)ether (BMEE; 0.625 mL) was added to 25 mL of deionised water in a 100 mL Duran bottle under stirring to give a 2.5% v/v solution. Tetrabutylammonium fluoride (92 mg) was added and the pH was adjusted to 9.6. A sheet of Fibro VS was patted dry with absorbing material and placed into a sealable tray. The solution of dithiol was added to the sheet, and the sealed tray was placed in a sealed bag to gently shake on an orbital shaker in a fumehood for 16 hrs. After this time, solution was decanted off in bleach, replaced with 50 mL deionised water and placed back on the orbital shaker for 20 minutes. This rinsing process was repeated four times before removing the last water wash.

### Addition of 2-Vinylpyridine to Fibro-BMEE

A 0.1 M phosphate buffer at pH 8 was prepared by dissolving K₂HPO₄ (16.73 g) and KH₂PO₄ (0.523 g) in 900 mL of deionised water, then adding deionised water QSAD for 1000 mL. A sheet of Fibro-linker-SH, obtained above, was placed in a polypropylene tray and rinsed twice with the freshly prepared phosphate buffer pH 8 before being patted dry. The sheet was then treated in a fumehood by addition of 25 ml of phosphate buffer pH 8, followed by 80 μL of 2-vinylpyridine to give a 30 mM solution. The polypropylene tray was closed with a lid then placed in a large zip bag, then sealed. The system was shaken overnight at room temperature (20-23° C.). The reaction solution was decanted in bleach and the sheet was rinsed with DI water (6×20 min) before being patted dry and tested.

### Thiophilic Chromatography

The above-described functionalized matrix materials were used as thiophilic interaction chromatography (TIC) media in the following experiments when isolating supercoiled plasmid DNA from other components, RNA, in the solution.

Membrane volumes of 0.4 mL were used, running at 5 MV/min equating to a flow rate of 2 ml/min, or a residence time of 12 seconds.

The binding buffer used comprised 2.0 M ammonium sulfate, 100 mM Tris, and 10 mM EDTA, at pH 7.5 containing the impure plasmid sample.

The wash buffer used comprised of 2.0 ammonium sulfate, 100 mM Tris, 10 mM EDTA, at pH 7.5 this was passed through the unit for 10 MV.

The elution buffer used comprised of 1.7 M ammonium sulfate, 100 mM Tris, 10 mM EDTA, 300 mM NaCl this was passed through the unit for 20 MV.

The strip buffer used to strip the column of remaining bound components comprised of 100 mM Tris, 10 mM EDTA at pH 7.5 and was passed through the unit for 15 MV.

The cleaning in place (CiP) solution was 0.5 M NaOH, this was left for 1 hour in contact with the unit.

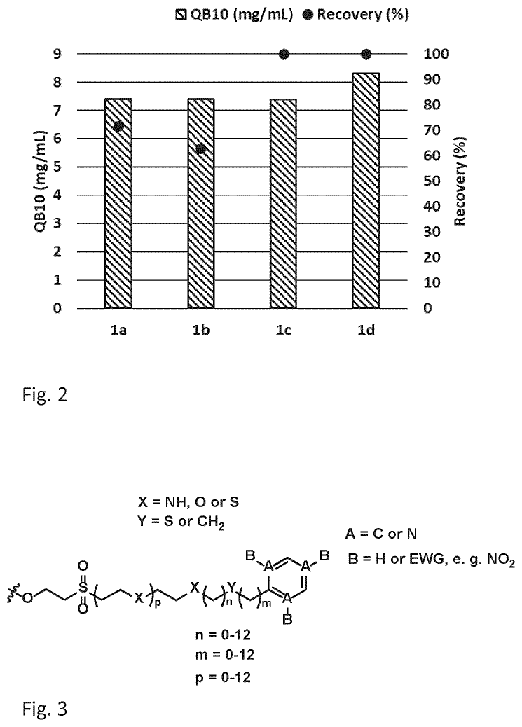

From the resulting chromatogram, which is illustrated in FIG. 4, a dynamic binding capacity (DBC) result can be calculated. The chromatogram demonstrates the separation of plasmid DNA in a solution from RNA.

The functionalization of matrix material described above using different ligand concentrations at start of reaction, 0.28, 0.14 and 0.07 mmol/mL, respectively, resulted in an approximate ligand density of 923, 622 and 331 μmol/g, respectively (see Table 1).

TABLE 1

| Ligand concentration | Approx. | Dynamic Binding | Dynamic Binding |
|---|---|---|---|
| at start of reaction | ligand density | Capacity (mg/ml) | Capacity (mg/ml) |
| (mmol/mL) | (μmol/g) | Plasmid (4.3 kb) | Herring Sperm DNA |
| 0.28 | 923 +/− 3 | 4.68 | 8.40 |
| 0.14 | 622 +/− 3 | 4.56 | 7.47 |
| 0.07 | 331 +/− 3 | 2.83 | 8.93 |

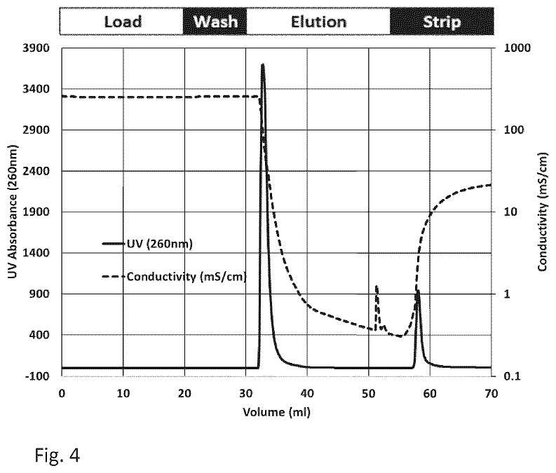

These different matrix materials with different ligand densities were then tested in different chromatography runs with analytes comprising a range of different sized plasmids (small 4.3 kb, mid-sized 6.1 kb and 11.6 kb, large 17.0 kb). In FIG. 5 is shown that the lowest ligand density (331 μmol/g) showed the lowest recovery with the small (64%) and mid-sized (88%) plasmids but better recovery for the largest (50%) plasmid in the series. The highest ligand density had the best recovery with all small and mid-sized plasmids, demonstrating the ligand density can be tuned to suit the target molecule size.

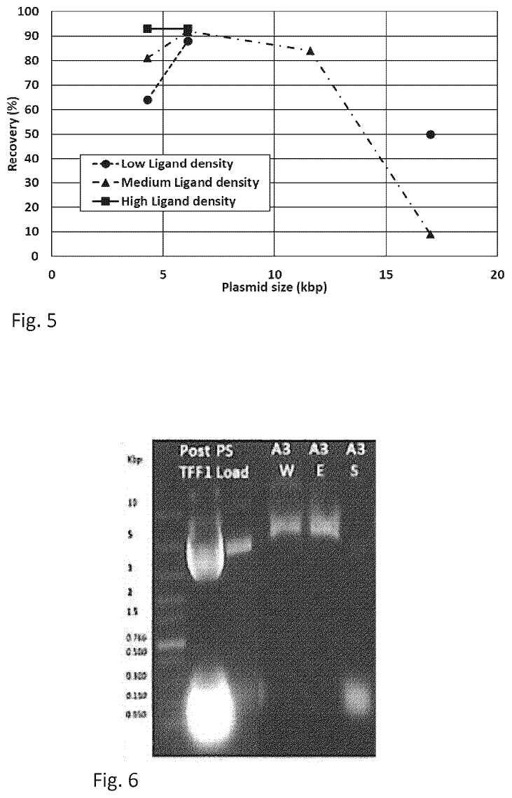

The quality of the thus purified plasmid was investigated next with the eluate from the tests run on a gel electrophoresis. This showed that the elution fraction was enriched with supercoiled DNA (FIG. 6). FIG. 6 shows an agarose gel loaded with sample from the wash, (A3 W in FIG. 6), the elution (A3 E in FIG. 6) and the strip phases (A3 S in FIG. 6). The gel was also loaded with a sample filtered with tangential flow filtration (TFF, with a 300 kDa cut-off) (Post TFF1 in FIG. 6), and with a sample as added to the chromatography column (PS Load in FIG. 6).

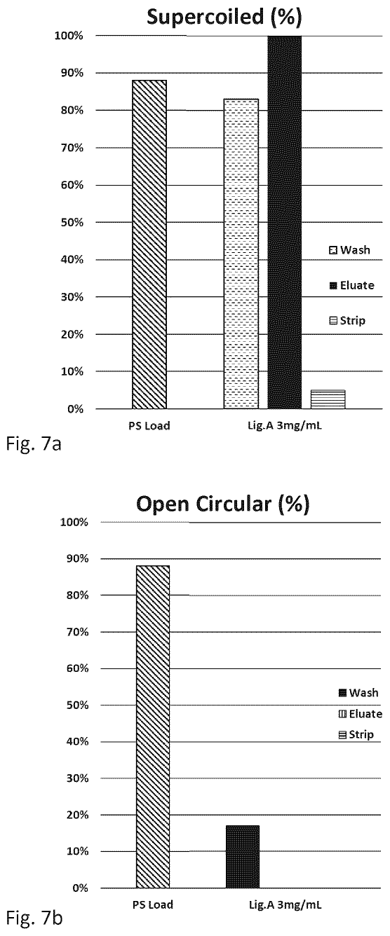

The elution fraction had a reduced impurity profile when compared with the starting feed (PS load), see FIGS. 7a-7b, which is summarised in table 2. Genomic DNA is reduced 10-fold, while RNA contaminates are reduced to 0.1 μg/mL. The fraction of super coiled DNA increases from 85% in the load to 95% in the eluate, see FIG. 7a, while the fraction of open circle DNA reduces to below limit of detection (LOD), see FIG. 7b, confirming the selectivity of the above described matrix material to supercoiled plasmid.

TABLE 2

| | Starting | Eluate from |
|---|---|---|
| | feed | chromatography |
| Genomic DNA (ng/ml) | 3000 | 300 |
| RNA (μg/ml) | 50 | 0.1 |
| RNA (w/w) | 35% | >1% |
| Plasmid Supercoiled (%) | 85% | 95% |
| Plasmid open circular (%) | ~5% | Below LOD |

The stabilities of the ligands from all routes shown in FIGS. 1a-1d were investigated in both basic and acidic buffers by exposing the functionalized chromatography material for these acidic/basic conditions for periods of from less than 20 min up to 4 hours. As indicated in Table 3, the dynamic binding capacity (DBC at 10% breakthrough) was tested using Herring Sperm DNA both before and after basic/acid buffer exposure.

TABLE 3

| | DBC before | DBC after |
|---|---|---|
| | (mg/ml) | (mg/ml) |
| 0.5M NaOH & 40% IPA (4 hrs) | 5.9 | 6.0 |
| 0.5M HCl (20 min) | 6.4 | 6.3 |
| 0.5M Phosphoric acid (20 min) | 6.3 | 6.4 |

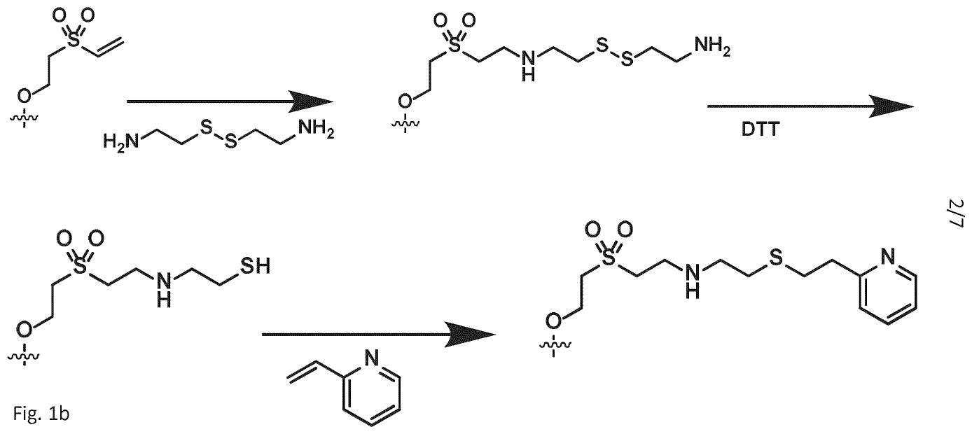

No drop in performance was observed, demonstrating the chemical stability of all the chemical routes. A shown in FIG. 2, all ligands produced from the described routes demonstrated a DBC between 7-8 mg/ml with Herring Sperm DNA, with high recoveries, base stability and low pressures at 8 MV/min.

### DISCUSSION

The chemistry routes described above, and illustrated in FIGS. 1-1d, produce a matrix material for a chromatographic medium capable of purifying and separating polynucleotide species via thiophilic interaction chromatography. The length of the spacer used in the chemistry route produces a product capable of surviving repeated and prolonged exposure to harsh conditions, included but not limited to cleaning solutions of 0.1-2.0 M NaOH or 0.1-0.5 M HCl or phosphoric acid.

This matrix material is capable of purifying different isoforms of plasmid from impurities such as RNA.

Combining the high DBCs with the short residence times, a productivity can be obtained that measures grams of plasmid per unit volume per unit time by tenfold more than what may be obtained with a resin equivalent.

1. A chromatography medium for separating an analyte in a solution, the chromatography medium comprising a matrix material having a mean flow pore size of 0.1-2.0 μm, wherein the matrix material is functionalized with a thiophilic aromatic ligand to a ligand concentration up to 1500 μmol/g matrix material.

2. The chromatography medium of claim 1, wherein the thiophilic aromatic ligand comprises an aryl group selected from pyridyl, phenyl, benzyl, toluyl, phenethyl, naphtyl, imidazolyl, pyrazolyl, pyrazinyl, pyrimidinyl, pyridazinyl, piperidinyl, morpholinyl, piperazinyl, indolyl, quinolinyl, purinyl groups and any substituted such group.

3. The chromatography medium of claim 1, wherein the matrix material M is functionalized with an aromatic ligand according to the formula: 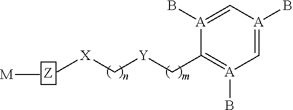
wherein, independently
M represents the matrix material,
Z represents a linker, optionally including a spacer moiety,
X for each occurrence is NH, O or S,
Y is S or CH₂, or is absent,
A is C or N,
B is H, a C1-C6 alkyl group such as a C1-C3 alkyl group, or an electron withdrawing group, or is absent,
n is 0 or an integer 1-12, and
m is 0 or an integer 1-12.

4. The chromatography medium of claim 1, wherein
the ligand or a portion of
the ligand is described by the formula: 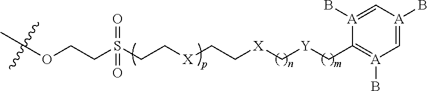
wherein
X for each occurrence is NH, O or S,
Y is S or CH₂,
A is C or N,
B is H or an electron withdrawing group, or B is absent,
n is 0 or an integer 1-12,
m is 0 or an integer 1-12, and
p is 0 or an integer 1-12.

5. The chromatography medium of claim 1, wherein the thiophilic aromatic ligand is selected from 2-mercaptopyridine, 4-mercaptopyridine, vinyl pyridine, 2-mercaptoethanol and 2-mercapto-ethyl-pyridine.

6. The chromatography medium of claim 1, wherein the ligand is connected to the matrix material through an extender group selected from polysaccharide structures and polymeric structures.

7. The chromatography medium of claim 1, wherein the matrix material is a convective flow material.

8. The chromatography medium of claim 1, wherein the matrix material is a non-woven fibrous material.

9. A chromatography unit comprising the chromatography medium of claim 1.

10. A process of separating an analyte in a solution, the process comprising:
obtaining a solution comprising an analyte,
adding the solution to the chromatography medium of claim 1 in a binding buffer having a conductivity of 240-270 mS/cm,
eluting the analyte from the chromatography medium by contacting the chromatography medium with an elution buffer having a conductivity of 0-240 mS/cm, and
collecting the thus formed eluate containing the analyte.

11. A process of separating different isoforms of DNA in a solution, the process comprising obtaining a solution comprising isoforms of plasmid DNA in a solution,
adding the solution to the chromatography medium of claim 1 in a binding buffer having a conductivity of 240-270 mS/cm,
contacting the chromatography medium with a first elution buffer having a conductivity of 200-240 mS/cm,
collecting the thus formed eluate containing a first plasmid DNA isoform, and thereafter contacting the chromatography medium with a second elution buffer having a conductivity of 0-200 mS/cm, and which conductivity is lower than the conductivity of the first elution buffer,
collecting the thus formed eluate containing a second plasmid DNA isoform.

12. The process of claim 10, wherein eluting the analyte from the chromatography medium comprises a stepwise or gradient elution in which the chromatography medium is contacted with elution buffers of decreasing conductivity.

13. The process of claim 11, wherein the second plasmid DNA isoform is supercoiled plasmid DNA.

14. The process of claim 10, further comprising, after the eluting step, a step of adding the eluate to a multimodal chromatography resin.

15. The process of claim 10, comprising cleaning of the chromatography medium after collecting the eluate, the cleaning comprising to add a solution of 0.1-2.0 M NaOH to the chromatography medium.

16. The process of claim 10, comprising cleaning of the chromatography medium after collecting the eluate, the cleaning comprising to add a solution of 0.1-0.5 M HCl or phosphoric acid to the chromatography medium.

17. Use of the chromatography medium of claim 1 or of the chromatography unit for separation of different isoforms of plasmid DNA present in a solution.

18. Use of the chromatography medium of claim 1 or of the chromatography unit for purification of supercoiled plasmid DNA.
